(19) United States
(12) Patent Application Publication
(10) Pub. No.: US 2009/0254150 A1
(43) Pub. Date: Oct. 8, 2009

(54) Electrical Stimulation of the Acoustic Nerve with Coherent Fine Structure

(75) Inventor: Clemens M. Zierhofer, Kundl (AT)

Correspondence Address: Sunstein Kann Murphy & Timbers LLP, 125 SUMMER STREET, BOSTON, MA, 02110-1618, US

(73) Assignee: MED-EL ELEKTROMEDIZINISCHE GERAETE GMBH, Innsbruck (AT)

(21) Appl. No.: 12/420,179

(22) Filed: Apr. 8, 2009

Related U.S. Application Data
(60) Provisional application No. 61/043,170, filed on Apr. 8, 2008.

(51) Int. Cl. A61F 11/04; A61N 1/36

(52) U.S. Cl. 607/57

(57) ABSTRACT

A method of enhancing temporal cues in a cochlear implant system is presented. The cochlear implant system includes an electrode array in which each electrode is stimulated based on a stimulation sequence of pulses. The method includes deriving signal c(t) from an acoustic representative electrical signal, the signal c(t) including low frequency temporal information. An estimate of spectral energy e(t) is derived from the acoustic representative electrical signal, the signal e(t) including spectral information with substantially no pitch related temporal information. The stimulation sequence is created for at least one electrode in the array as a function of c(t) and e(t).

### CROSS-REFERENCE TO RELATED APPLICATIONS

This application claims priority from U.S. provisional application No. 61/043,170 filed Apr. 8, 2008, which is incorporated herein by reference.

### TECHNICAL FIELD

The present invention relates to electrical stimulation of the acoustic nerve, and more particularly to a coherent fine structure approach for a cochlear implant.

### BACKGROUND ART

Cochlear implants are a possibility to help profoundly deaf or severely hearing impaired persons. Unlike conventional hearing aids, which just apply an amplified and modified sound signal, a cochlear implant is based on direct electrical stimulation of the acoustic nerve. The intention of a cochlear implant is to stimulate nervous structures in the inner ear electrically in such a way that hearing impressions most similar to normal hearing are obtained.

A normal ear transmits sounds as shown in FIG. 1 through the outer ear 101 to the eardrum 102, which moves the bones of the middle ear 103, which in turn excites the cochlea 104. The cochlea 104 includes an upper channel known as the scala vestibuli 105 and a lower channel known as the scala tympani 106, which are connected by the cochlear duct 107. In response to received sounds transmitted by the middle ear 103, the fluid filled scala vestibuli 105 and scala tympani 106 function as a transducer to transmit waves to generate electric pulses that are transmitted to the cochlear nerve 113, and ultimately to the brain.

Cochlear implant systems have been developed to overcome this by directly stimulating the user's cochlea 104. A cochlear implant system typically includes two parts, the speech processor and the implanted stimulator. The speech processor (not shown in FIG. 1) may include the power supply (batteries) of the overall system, and a microphone that provides an audio signal input to an external signal processing stage where various signal processing schemes can be implemented. The processed audio signal is then converted into a digital data format, such as a sequence of data frames, for transmission into receiver 108 of the implanted stimulator.

The connection between speech processor and the receiver 108 of the implanted stimulator is established either by means of a radio frequency link (transcutaneous) or by means of a plug in the skin (percutaneous). FIG. 1 shows a typical arrangement based on inductive coupling through the skin to transfer both the required electrical power and the processed audio information. As shown in FIG. 1, an external transmitter coil 111 (coupled to the external signal processor) is placed on the skin adjacent to a subcutaneous receiver coil 112 (connected to the receiver 108). Often, a magnet in the external coil structure interacts with a corresponding magnet in the subcutaneous secondary coil structure. This arrangement inductively couples a radio frequency (rf) electrical signal to the receiver 108. The receiver 108 is able to extract from the rf signal both the audio information for the implanted portion of the system and a power component to power the implanted system.

Besides extracting the audio information, the receiver 108 also performs additional signal processing such as error correction, pulse formation, etc., and produces a stimulation pattern (based on the extracted audio information) that is sent through connected wires 109 to an implanted electrode carrier 110. Typically, this electrode carrier 110 includes multiple electrodes on its surface that provide selective stimulation of the cochlea 104.

At present, the most successful stimulation strategy is the so called “continuous-interleaved-sampling strategy” (CIS) introduced by Wilson B. S., Finley C. C., Lawson D. T., Wolford R. D., Eddington D. K., Rabinowitz W. M., “Better speech recognition with cochlear implants,” Nature, vol. 352, 236-238, July 1991B, which is incorporated herein by reference in its entirety. Signal processing for CIS in the speech processor involves the following steps:

1) Splitting up of the audio frequency range into spectral bands by means of a filter bank;

2) Envelope detection of each filter output signal;

3) Instantaneous nonlinear compression of the envelope signals (map law); and,

(4) Adaptation to thresholds (THR) and most comfortable loudness (MCL) levels.

According to the tonotopic organization of the cochlea, each stimulation electrode in the scala tympani is associated with a band-pass filter of the external filter bank. For stimulation, symmetrical biphasic current pulses are applied. The amplitudes of the stimulation pulses are directly obtained from the compressed envelope signals (step (3) of above). These signals are sampled sequentially, and the stimulation pulses are applied in a strictly non-overlapping sequence. Thus, as a typical CIS-feature, only one stimulation channel is active at one time.

CIS has proven to be very successful in conveying speech information, in particular for western languages such as, e.g., English, French, etc. However, some potential to improve the performance of cochlear implants can be found in the field of tonal languages such as, e.g., Mandarin, Vietnamese, etc., and in the field of music perception. In both fields, a lot of information is contained in the so called fundamental frequency, sometimes designated as the pitch frequency, and temporal variations thereof. With CIS, the fundamental frequency is only weakly represented in the temporal patterns of stimulation pulses.

### SUMMARY OF THE INVENTION

In accordance with one embodiment of the invention, a method of enhancing temporal cues in a cochlear implant system is presented. The cochlear implant system includes an electrode array in which each electrode is stimulated based on a stimulation sequence of pulses. The method includes deriving signal c(t) from an acoustic representative electrical signal, the signal c(t) including low frequency temporal information. An estimate of spectral energy e(t) is derived from the acoustic representative electrical signal, the signal e(t) including spectral information with substantially no pitch related temporal information. The stimulation sequence is created for at least one electrode in the array as a function of c(t) and e(t).

In accordance with related embodiments of the invention, creating the stimulation signal may include multiplying e(t) by c(t). Deriving the estimate of spectral energy e(t) may include applying the acoustic representative electrical signal to a bank of filters, each filter in the bank of filters associated with a channel that includes an electrode in the electrode array, the number of channels equal to N. The spectral energy is estimated for each channel after filtering to form eᵢ(t), (i=1, 2, . . . , N).

In accordance with further related embodiments of the invention, deriving signal c(t) may include filtering the acoustic representative electrical signal to form signal x(t), performing half wave rectification on x(t) to form signal xₕ(t), and performing amplitude normalization on xₕ(t) to form the signal c(t). Filtering may include band-pass filtering, for example, between 80 Hz to 400 Hz. Performing amplitude normalization may include performing peak detection on x(t) to form peak detector signal xₚ(t), and dividing xₕ(t) by xₚ(t) to form the signal c(t). Performing amplitude normalization may include deriving Hilbert envelope env(x(t)) of x(t), and dividing xₕ(t) by the env(x(t)) to form signal c(t). Performing amplitude normalization may include dividing xₕ(t) by x_{power}(t) wherein x_{power}(t) represents the instantaneous power of signal x(t).

In accordance with still further related embodiments of the invention, deriving signal c(t) may include filtering the acoustic representative electrical signal to form signal x(t), and associating segments x(t)>0 to amplitude c(t)=1, and segments x(t)<0 to amplitude c(t)=0.

In accordance with further embodiments of the invention, deriving signal c(t) may include using a pitch picker.

In accordance with yet further related embodiments of the invention, the method may further include applying the acoustic representative electrical signal to a bank of filters, each filter in the bank of filters associated with a channel that includes an electrode in the electrode array, the method further comprising setting c(t) equal to one for at least one channel filtered at the high frequency end. For example, c(t) may be set to one for channels covering a range higher than 1 kHz.

In accordance with another embodiment of the invention, a system for enhancing temporal cues in a cochlear implant system is presented. The cochlear implant system includes an electrode array in which each electrode is stimulated based on a stimulation sequence of pulses. A first module derives signal c(t) from an acoustic representative electrical signal, the signal c(t) including low frequency temporal information. A second module estimates spectral energy e(t) from the acoustic representative electrical signal, the signal e(t) including spectral information with substantially no pitch related temporal information. A third module creates the stimulation sequence for at least one electrode in the array as a function of c(t) and e(t).

In accordance with related embodiments of the invention, the third module may include a multiplier for multiplying c(t) and e(t). The second module may include a band of filters for filtering the acoustic representative electrical signal, each filter in the bank of filters associated with a channel that includes an electrode in the electrode array, the number of channels equal to N. An estimator estimates spectral energy for each channel after filtering to form eᵢ(t), (i=1, 2, . . . , N).

In accordance with further related embodiments of the invention, the first module includes a band-pass filter for filtering an acoustic representative electrical signal to form signal x(t). A half-wave rectifier performs half wave rectification on x(t) to form signal xₕ(t). A normalizer performs amplitude normalization on xₕ(t) to form signal c(t). The band-pass filter may pass signals between 80 Hz to 400 Hz. The normalizer may include a peak detector for forming peak detector signal xₚ(t); and a divider module for dividing xₕ(t) by xₚ(t) to form the signal c(t). The normalizer may include a Hilbert module for deriving Hilbert envelope env(x(t)) of x(t), and a divider module for dividing xₕ(t) by env(x(t)) to form signal c(t). The normalizer may include a divider for dividing xₕ(t) by x_{power}(t), wherein x_{power}(t) represents the instantaneous power of signal x(t).

In accordance with yet further related embodiments of the invention, the system includes a filter for filtering the acoustic representative electrical signal, wherein the first module includes an association module for associating segments x(t)>0 to amplitude c(t)=1, and segments x(t)<0 to c(t)=0.

In accordance with further related embodiments of the invention, the first module may include a pitch picker.

In accordance with still further embodiments of the invention, the system may include a bank of filters for filtering the acoustic representative electrical signal, each filter in the bank of filters associated with a channel that includes an electrode in the electrode array, wherein the first module sets c(t) equal to one for at least one channel filtered at the high frequency end. For example, the first module may set c(t) equal to one for channels filtered at higher than 1 kHz. For example, the first module may set c(t) to one for channels covering a range higher than 1 kHz.

In accordance with yet another embodiment of the invention, a computer program product for enhancing temporal cues in a cochlear implant system is presented. The cochlear implant system includes an electrode array in which each electrode is stimulated based on a stimulation sequence of pulses. The computer program product includes a computer usable medium having computer readable program code thereon. The computer readable program code includes program code for deriving signal c(t) from an acoustic representative electrical signal, the signal c(t) including low frequency temporal information. The computer readable program code further includes program code for deriving an estimate of spectral energy e(t) from the acoustic representative electrical signal, the signal e(t) including spectral information with substantially no pitch related temporal information. The computer readable program code still further includes program code for creating the stimulation sequence for at least one electrode in the array as a function of c(t) and e(t).

### BRIEF DESCRIPTION OF THE DRAWINGS

The foregoing features of the invention will be more readily understood by reference to the following detailed description, taken with reference to the accompanying drawings, in which:

FIG. 1 shows elements of a typical cochlear implant system and relevant ear structures;

FIGS. 2 A-C show generation of carrier c(t), in accordance with various embodiments of the invention;

FIGS. 3 A-D show generation of a CFS signal derived from band filter no. 1[350 Hz-550 Hz] of a 6-channel system, in accordance with various embodiments of the invention;

FIGS. 4 A-C show an exemplary CFS stimulation sequence and CIS stimulation sequence, derived from band filter No. 1[350 Hz-550 Hz] of a 6-channel system, in accordance with various embodiments of the invention. The stimulation pulse rate in both sequences is 3 kpulses/sec;

FIG. 5 shows exemplary CFS stimulation sequences of a 6-channel CFS system, assuming pulse rates of 3 kpulses/sec per channel, in accordance with various embodiments of the invention; and

FIG. 6 shows exemplary CIS stimulation sequences of a 6-channel CFS system, assuming pulse rates of 3 kpulses/sec per channel.

### DETAILED DESCRIPTION OF SPECIFIC EMBODIMENTS

In illustrative embodiments of the invention, a system and method of enhancing the representation of low frequency temporal cues associated with a cochlear implant is presented, and shall be referred to herein as the “Coherent Fine Structure (CFS)” approach. Details are discussed below.

The CFS approach is directed primarily at a better representation of temporal cues in the pitch frequency range, typically between, without limitation, 80 Hz and 400 Hz. Signal processing according to CFS may involve a filter bank, similar as for CIS. Illustratively, the overall frequency range of the audio signal may be split up by N band-pass filters, resulting in N output signals bᵢ(t) (i=1, 2, . . . , N). For each filter output, an estimate of spectral energy eᵢ(t) (i=1, 2, . . . , N) is determined. For example, signals eᵢ(t) may be r.m.s. signals of the filter output signals. It is assumed that signals eᵢ(t) are slowly varying with time, and typically do not include frequency components higher than about the lower limit of the pitch frequency range, typically about 80 Hz.

Temporal fine structure information is introduced by a carrier signal c(t). Carrier c(t) reflects the instantaneous pitch frequency directly as temporal information. Illustratively, carrier signal c(t) may vary, for example, between amplitudes zero and one. In preferred embodiments, c(t) is multiplied with each of the estimated spectral energy signals eᵢ(t) (i=1, 2, . . . , N). The product signals c(t)eᵢ(t) are used to derive the stimulation pulse amplitudes of the individual channels of an N-channel system. Since c(t) is applied coherently for all CFS channels, the temporal structure of the pitch frequency is generally not impaired by effects due to spatial channel interaction. With the CFS concept, a clear separation between “spectral information”—represented by the envelope signals eᵢ(t)—and “fine structure information”—represented by signal c(t)—is achieved.

An example of a carrier signal c(t), and a illustrative procedure to derive carrier signal c(t) from an audio signal, is as follows:

(1) Band-pass filtering of the audio signal in the range 80 Hz to 400 Hz (resulting in signal x(t));

(2) Half wave rectification (resulting in signal xₕ(t)); and

(3) Amplitude normalization (resulting in signal c(t)).

Amplitude normalization (step (3)) may be achieved, without limitation, by utilizing a peak detector. The peak detector signal xₚ(t) tracks the positive peaks of x(t), and in between two peaks, xₚ(t) is decaying with a particular time constant τ. Typically, τ is in the range of some tens of milliseconds. Carrier c(t) is an “amplitude-normalized” version of xₕ(t), i.e., c(t)=xₕ(t)/xₚ(t). The purpose of c(t) is essentially to preserve the temporal structure of x(t).

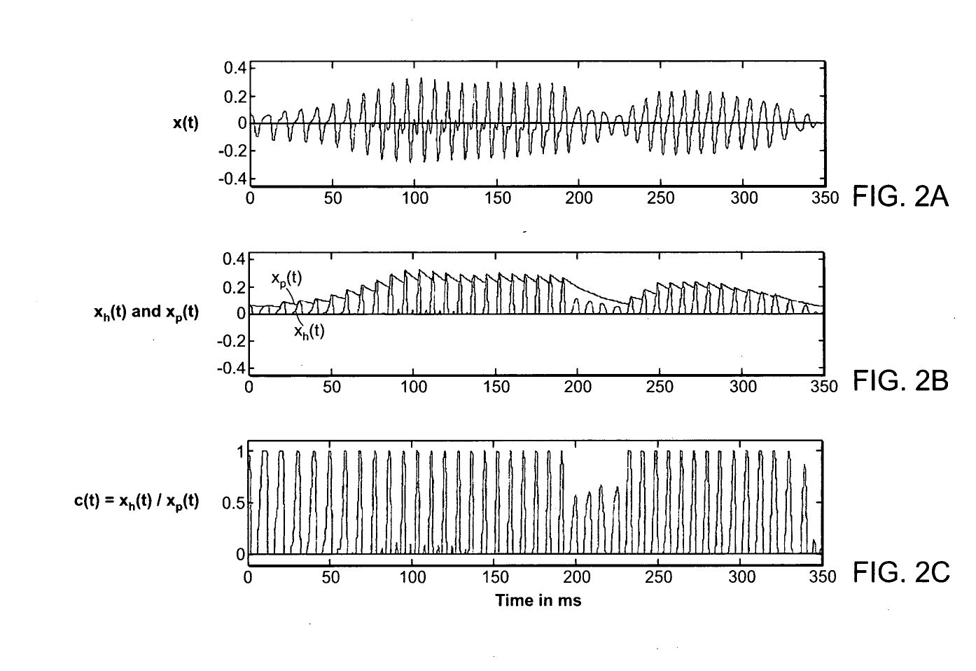

FIGS. 2 A-C show the generation of carrier c(t), in accordance with various embodiments of the invention. FIG. 2A shows a band-pass filtered version x(t) of a voiced speech sample. The mean amplitude is strongly varying. FIG. 2B shows the half wave rectified version xₕ(t), and the peak picker signal xₚ(t). The ratio c(t)=xₕ(t)/xₚ(t) is depicted in FIG. 2C. The amplitude of c(t) is smaller than 1, whenever amplitude reductions in x(t) occur which are faster than the time constant τ. In FIG. 2 this occurs, for example, at about t≈200 to 230 ms.

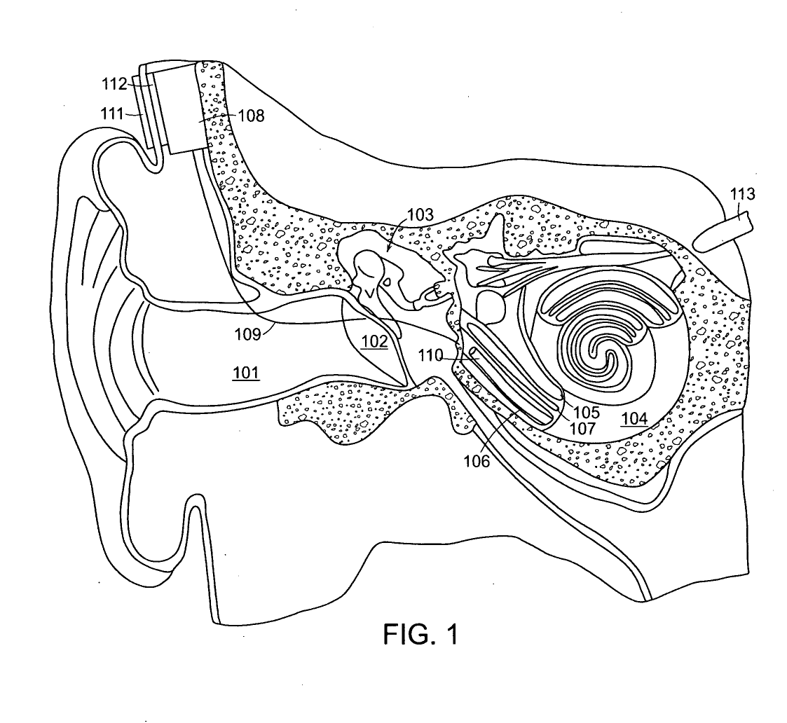

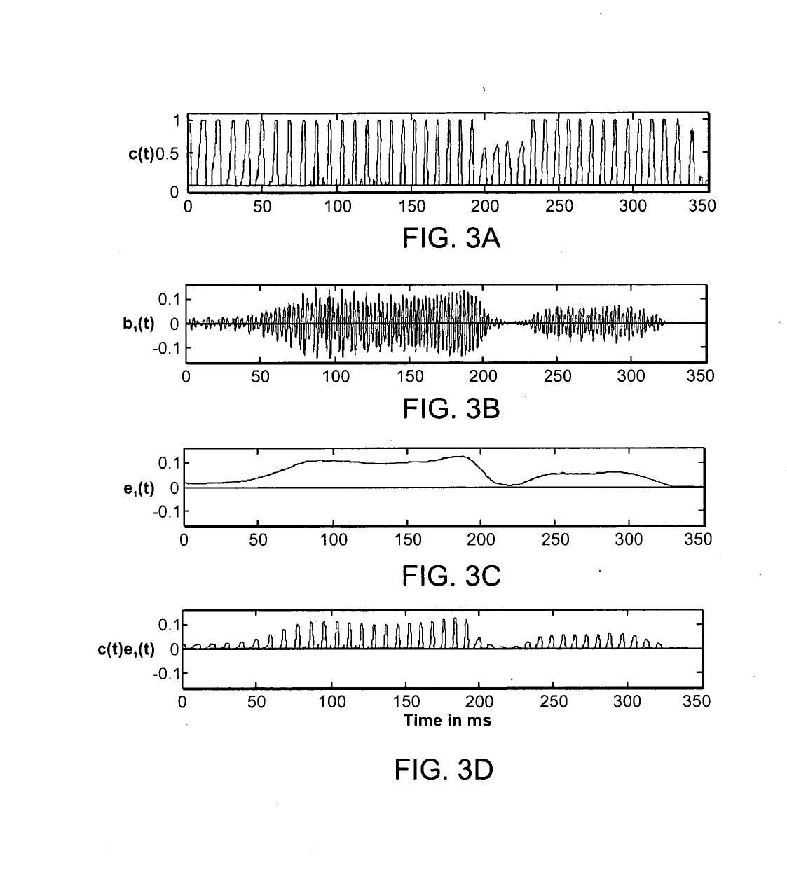

FIGS. 3A-D shows an example of signals appearing in channel No. 1 of a 6-channel stimulation system, in accordance with various embodiments of the invention. More particularly, FIG. 3A depicts carrier c(t) as derived in FIG. 1. FIG. 3B depicts the band-pass output signal b₁(t) of a band-pass filter within [350 Hz-550 Hz]. FIG. 3C depicts an estimate of spectral energy e₁(t) associated with output signal b₁(t). Obviously, e₁(t) is slowly varying with time and free of rapid temporal fluctuations. In particular, the pitch frequency is not emphasized in e₁(t). FIG. 3D depicts the product signal c(t)e₁(t). This signal now includes both the temporal fine structure of c(t) and the spectral information of e₁(t).

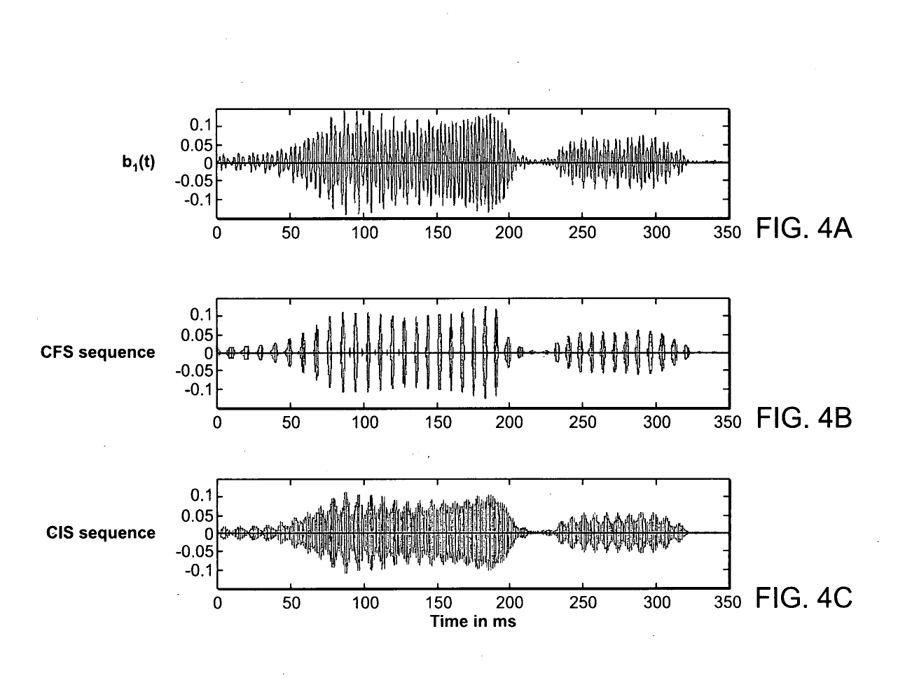

FIGS. 4 A-C is a comparison of the CIS and CFS approaches. FIG. 4A shows the band-pass output signal b₁(t) of a band-pass filter within [350 Hz-550 Hz]. FIG. 4B depicts the resulting CFS sequence of stimulation pulses at a rate of 3 kpulses/sec, which is the result of sampling the CFS signal shown in FIG. 3C, in accordance with various embodiments of the invention. Each vertical line represents a biphasic stimulation pulse. The pitch frequency is clearly represented by the CFS signal in FIG. 4B. FIG. 4C depicts the resulting CIS-sequence at a rate of 3 kpulses/sec, representing the envelope of b₁(t). Obviously, the variations in the pulse amplitudes in the CIS sequence provide pitch frequency information. However, in contrast to CFS, this representation is much less pronounced.

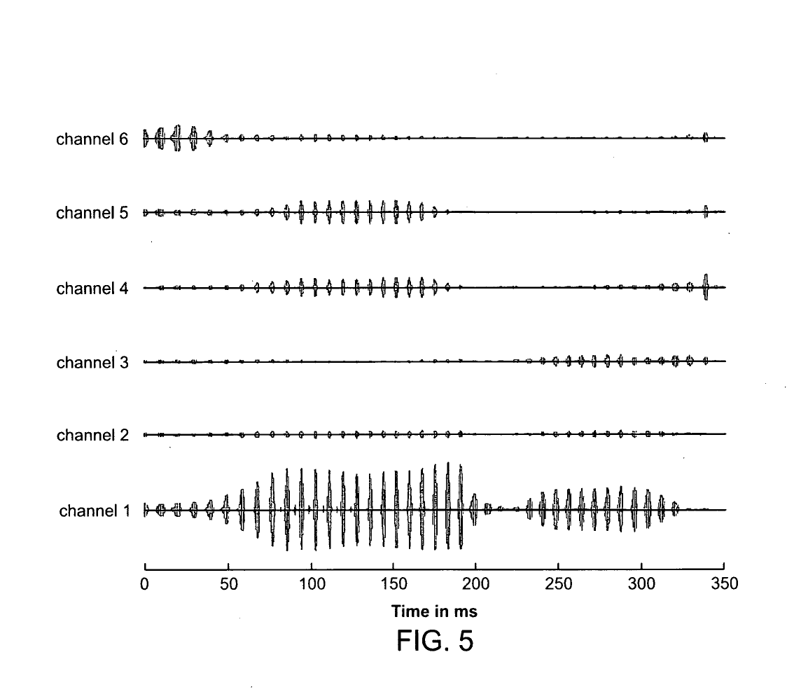

FIG. 5 shows an example of stimulation sequences of a 6-channel system with an overall frequency range is [350 Hz-5500 Hz], in accordance with various embodiments of the invention. All channels, with channel 1 derived from the lowest band filter, and channel 6 derived from the highest band filter, are depicted. The overall pulse rate here is 18 kpulses/sec, resulting in non-overlapping pulses at repetition rates of 3 kpulses/sec for each channel. The individual stimulation sequences represent sampled versions of product signals c(t)eᵢ(t). As intended, the pitch frequency is clearly represented by the sequences of pulse bursts in the individual channels. Since the pulse bursts apply coherently in time across the channels, the pitch representation is very robust against influences due to spatial channel interaction.

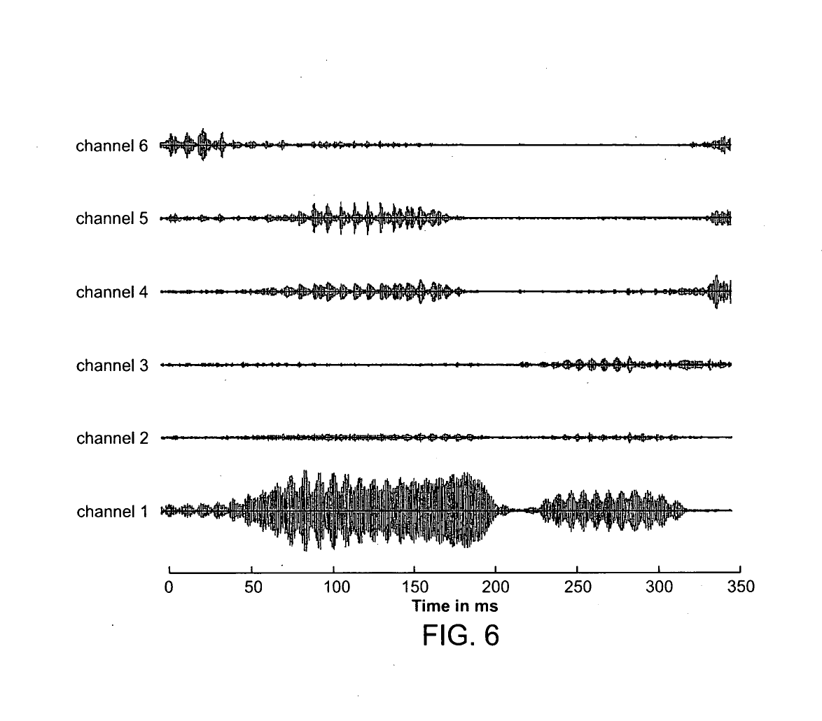

FIG. 6 presents a 6-channel CIS representation. The same voiced speech segment and the same filter bank as for the CFS representation of FIG. 4 is used. Obviously, the pitch structure is reflected by a more or less pronounced amplitude modulation. However, the overall representation of pitch is much clearer in the CFS than in the CIS representation.

The CFS concept primarily concerns audio signals where a clear pitch component is present, e.g., voiced speech segments. In various embodiments, situations without a clear pitch component may be detected, for example, by means of a voiced/unvoiced detector, and the carrier c(t) may be set to c(t)=1. Then, product signals c(t)eᵢ(t) are equal to eᵢ(t), and hence are represented by stimulation pulses at the rate equal to the frame rate per channel.

The representation of c(t)eᵢ(t) by means of stimulation pulses can theoretically be achieved by utilizing a sufficiently high pulse repetition rate, However, if the overall pulse repetition rate for an adequate temporal resolution of signals c(t)eᵢ(t) would be too high, supporting concepts such as: “Channel Interaction Compensation (CIC)” (for simultaneous stimulation) as described in Zierhofer C. M., “Electrical nerve stimulation based on channel specific sampling sequences,” U.S. Pat. No. 6,594,525, 2003; and/or the “Selected Group (SG)” algorithm, as described in Zierhofer C. M., “Electrical stimulation of the acoustic nerve based on selected groups,” U.S. Patent Application 20050203589 (pending) can be utilized. Each of these documents is incorporated herein by reference in their entirety. Note that while the CSSS approach as described in U.S. Pat. No. 6,594,525 clearly enhances the temporal fine structure in the individual channels, the fine structure is not presented coherently.

In practical applications, the low frequency information may be removed for channels at the high frequency end, by setting c(t) equal to one. This prevents low frequency temporal information from being in conflict with the frequency which is associated with the electrode position (tonotopic principle). For example, c(t) may be set to one for channels covering a range higher than 1 kHz. For these particular channels, the stimulation is similar to CIS.

In various embodiments, carrier c(t) may be obtained by xₕ(t)/env(x(t)), where xₕ(t) is the half wave rectified version of the band-pass filtered audio signal x(t), and env(x(t)) is its Hilbert envelope. Still another method to obtain carrier c(t) may be to simply associate segments x(t)>0 to amplitude c(t)=1, and segments x(t)<0 to c(t)=0. In this case, only the zero crossings of x(t) are used to encode the temporal fine structure. Still yet another method to obtain carrier c(t) may be to compute c(t)=xₕ(t)/x_{power}(t), where x_{power}(t) is an estimate of the instantaneous power of signal x(t).

Another method to obtain a carrier signal c(t) may be based on a pitch picker. Examples of pitch pickers are described, without limitation, in W. Hess, “Pitch determination of speech signals,” Ed. Springer, Berlin, 1983, which is incorporated herein by reference.

In various embodiments, the band-pass filtered version x(t) of the audio signal may cover the range of about [100 Hz-1000 Hz], covering the frequency ranges pitch—and first format frequency.

In various embodiments, the disclosed method may be implemented as a computer program product for use with a computer system. Such implementation may include a series of computer instructions fixed either on a tangible medium, such as a computer readable media (e.g., a diskette, CD-ROM, ROM, or fixed disk) or transmittable to a computer system, via a modem or other interface device, such as a communications adapter connected to a network over a medium. Medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable media with accompanying printed or electronic documentation (e.g., shrink wrapped software), preloaded with a computer system (e.g., on system ROM or fixed disk), or distributed from a server or electronic bulletin board over the network (e.g., the Internet or World Wide Web).

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A method of enhancing temporal cues in a cochlear implant system, the cochlear implant system including an electrode array in which each electrode is stimulated based on a stimulation sequence of pulses; the method comprising:
deriving signal c(t) from an acoustic representative electrical signal, the signal c(t) including low frequency temporal information;
deriving an estimate of spectral energy e(t) from the acoustic representative electrical signal, the signal e(t) including spectral information with substantially no pitch related temporal information; and
creating the stimulation sequence for at least one electrode in the array as a function of c(t) and e(t).

2. The method according to claim 1, wherein creating the stimulation signal includes multiplying e(t) by c(t).

3. The method according to claim 1, wherein deriving the estimate of spectral energy e(t) includes:
applying the acoustic representative electrical signal to a bank of filters, each filter in the bank of filters associated with a channel that includes an electrode in the electrode array, the number of channels equal to N; and
estimating spectral energy for each channel after filtering to form eᵢ(t),(i=1, 2, . . . , N).

4. The method according to claim 1, wherein deriving signal c(t) includes:
filtering the acoustic representative electrical signal to form signal x(t);
performing half wave rectification on x(t) to form signal xₕ(t); and
performing amplitude normalization on xₕ(t) to form the signal c(t).

5. The method according to claim 4, wherein filtering includes band-pass filtering.

6. The method according to claim 4, wherein band-pass filtering includes passing signals between 80 Hz to 400 Hz.

7. The method according to claim 4, wherein performing amplitude normalization includes:
performing peak detection on x(t) to form peak detector signal xₚ(t); and
dividing xₕ(t) by xₚ(t) to form the signal c(t).

8. The method according to claim 4, wherein performing amplitude normalization includes:
deriving Hilbert envelope env(x(t)) of x(t); and
dividing xₕ(t) by the env(x(t)) to form signal c(t).

9. The method according to claim 4, wherein performing amplitude normalization includes dividing xₕ(t) by x_{power}(t), wherein x_{power}(t) represents the instantaneous power of signal x(t).

10. The method according to claim 1, wherein deriving signal c(t) includes:
filtering the acoustic representative electrical signal to form signal x(t); and
associating segments x(t)>0 to amplitude c(t)=1, and segments x(t)<0 to amplitude c(t)=0.

11. The method according to claim 1, wherein deriving signal c(t) includes a pitch picker.

12. The method according to claim 1, further including:
applying the acoustic representative electrical signal to a bank of filters, each filter in the bank of filters associated with a channel that includes an electrode in the electrode array, the method further comprising setting c(t) equal to one for at least one channel filtered at the high frequency end.

13. The method according to claim 12, wherein c(t) is set to one for channels covering a range higher than 1 kHz.

14. A system for enhancing temporal cues in a cochlear implant system, the cochlear implant system including:
an electrode array in which each electrode is stimulated based on a stimulation sequence of pulses;
a first module for deriving signal c(t) from an acoustic representative electrical signal, the signal c(t) including low frequency temporal information;
a second module for estimating spectral energy e(t) from the acoustic representative electrical signal, the signal e(t) including spectral information with substantially no pitch related temporal information; and
a third module for creating the stimulation sequence for at least one electrode in the array as a function of c(t) and e(t).

15. The system according to claim 14, wherein the third module includes a multiplier for multiplying c(t) and e(t).

16. The system according to claim 14, further comprising:
a band of filters for filtering the acoustic representative electrical signal, each filter in the bank of filters associated with a channel that includes an electrode in the electrode array, the number of channels equal to N, wherein the second module includes an estimator for estimating spectral energy for each channel after filtering to form eᵢ(t), (i=1, 2, . . . , N).

17. The system according to claim 14, wherein the first module includes:
a band-pass filter for filtering an acoustic representative electrical signal to form signal x(t);
a half-wave rectifier for performing half wave rectification on x(t) to form signal xₕ(t);
a normalizer for performing amplitude normalization on xₕ(t) to form signal c(t).

18. The system according to claim 17, wherein the band-pass filter passes signals between 80 Hz to 400 Hz.

19. The system according to claim 17, wherein the normalizer includes a peak detector for forming peak detector signal xₚ(t); and a divider module for dividing xₕ(t) by xₚ(t) to form the signal c(t).

20. The system according to claim 17, wherein the normalizer includes a Hilbert module for deriving Hilbert envelope env(x(t)) of x(t), and a divider module for dividing xₕ(t) by env(x(t)) to form signal c(t).

21. The system according to claim 17, wherein the normalizer includes a divider for dividing xₕ(t) by x_{power}(t) wherein x_{power}(t) represents the instantaneous power of signal x(t).

22. The system according to claim 14, further comprising a filter for filtering the acoustic representative electrical signal, wherein the first module includes an association module for associating segments x(t)>0 to amplitude c(t)=1, and segments x(t)<0 to amplitude c(t)=0.

23. The system according to claim 14, wherein the first module includes a pitch picker.

24. The system according to claim 14, further including a bank of filters for filtering the acoustic representative electrical signal, each filter in the bank of filters associated with a channel that includes an electrode in the electrode array, wherein the first module sets c(t) equal to one for at least one channel filtered at the high frequency end.

25. The system according to claim 24, wherein the first module sets c(t) equal to one for channels covering a range higher than 1 kHz.

26. A computer program product for enhancing temporal cues in a cochlear implant system, the cochlear implant system including an electrode array in which each electrode is stimulated based on a stimulation sequence of pulses the computer program product comprising a computer usable medium having computer readable program code thereon, the computer readable program code comprising:
program code for deriving signal c(t) from an acoustic representative electrical signal, the signal c(t) including low frequency temporal information;
program code for deriving an estimate of spectral energy e(t) from the acoustic representative electrical signal, the signal e(t) including spectral information with substantially no pitch related temporal information; and
program code for creating the stimulation sequence for at least one electrode in the array as a function of c(t) and e(t).
